(19) United States
(12) Patent Application Publication
(10) Pub. No.: US 2015/0377782 A1
(43) Pub. Date: Dec. 31, 2015

(54) Terahertz system

(71) Applicant: TOPTICA Photonics AG, Grafelfing (DE)

(72) Inventors: Nico Vieweg, Stockdorf (DE); Florian Rettich, Munchen (DE)

(73) Assignee: TOPTICA PHOTONICS AG, Grafelfing (DE)

(21) Appl. No.: 14/747,696

(22) Filed: Jun. 23, 2015

(30) Foreign Application Priority Data
Jun. 25, 2014 (DE) 102014009167.8

(51) Int. Cl. G01N 21/59; G01N 33/483; G01J 5/10

(52) U.S. Cl. CPC G01N 21/59; G01J 5/10; G01N 33/483

(57) ABSTRACT

The present invention relates to a terahertz system for generating and time-resolved incoherent detecting of THz radiation, said system comprising a pulse laser light source (1) emitting laser pulses with a pulse duration of up to 1 ps at a repetition frequency of at least 1 MHz, a first THz antenna serving as sender (3) which is optically coupled to the laser light source (1) and converts the laser pulses into THz pulses with a pulse duration of up to 10 ps, and a second THz antenna serving as receiver (5). It is the object of the present invention to provide an improved terahertz system. As compared with the state of the art, it is above all intended to allow for a faster incoherent measurement of THz radiation. To this end, the present invention proposes to couple the second THz antenna to a detector circuitry whose bandwidth is at least equivalent to the repetition frequency of the laser light source. Furthermore, the present invention relates to applications of the terahertz system as well to a method for generating and time-resolved incoherent detecting of THz radiation.

The present invention relates to a terahertz system for generating and time-resolved incoherent detecting of THz radiation, said system comprising
a pulsed laser light source emitting laser pulses with a pulse duration of up to 1 ps at a repetition frequency of at least 1 MHz, preferably at least 10 MHz,
a first THz antenna serving as sender, said antenna coupled optically to the laser light source and converting laser pulses into THz pulses, and
a second THz antenna serving as receiver.

Furthermore, the present invention relates to a method for generating and time-resolved incoherent detecting of THz radiation as well as to applications of the terahertz system.

Designated as THz radiation is electromagnetic radiation in a frequency range of approx. 0.1 to 10 THz. Since molecular vibrations of different substances occur in the frequency range of THz radiation, it is possible to investigate corresponding substances and also to identify certain chemical compounds by means of absorption spectroscopy in the THz range. Moreover, objects, for example, with a radiation in the THz range can be imaged or investigated tomographically (THz CT). Since THz radiation penetrates well through dielectric substances like paper or textiles, objects within wrappings (e.g. packages) can also be located. Hence, there is a scientific and economic as well as a safety-relevant interest in low-cost and efficient terahertz systems. A difficulty lies in that systems working with THz radiation are difficult to realize. The reason is that for electronic signal processing the frequency of the THz radiation is very high. Compared with frequencies occurring in photonoics, however, the frequency of THz radiation is very low.

It is known that THz radiation can be generated and substantiated with photoconductive sender and receiver antennae utilizing ultra-short laser pulses with pulse durations in a range of 1 ps (e.g. see U.S. Pat. No. 5,789,750). For coherent time-resolved THz spectroscopy, it is required to ensure by means of an adjustable delay of the laser pulses that the chronological progression of the THz radiation passing the relevant sample to be investigated can be scanned at the receiving antenna (so-called pump-probe technique).

A disadvantage lies in that prior art methods for time-resolved THz spectroscopy are limited in terms of measuring speed. The measuring time per data point ranges at some ms. This circumstance has hitherto prevented the use of terahertz systems in applications in which, for example for quality assurance, products are to be examined which are moved quickly (by several meters per second), for instance on a production line, past the sensorics (sender/receiver) of the terahertz system. Likewise critical hitherto due to limited measuring speed are applications in which a sample is to be surveyed not only at a selective point but also across a major surface area.

For terahertz systems with incoherent detection, state-of-the-art systems hitherto utilize Golay cells, helium-cooled bolometers or pyroelectric detectors for reception of THz radiation. With these detectors, however, the time resolution lies in a range of several 100 ms or even seconds. Moreover, pyroelectric detectors require costly senders with a high radiation output. Therefore, such systems are not at all suitable for fast time-resolved THz measurements.

Against this background, it is the object of the present invention to provide an improved system. Compared to the state of the art, the target is a faster incoherent measurement of THz radiation.

The present invention achieves this object based on a terahertz system of the initially mentioned type by coupling the second THz antenna to a detector circuitry whose bandwidth is at least equivalent to the repetition frequency of the laser light source.

Furthermore, the object is achieved by a method for generating and time-resolved incoherent detecting of THz radiation, said method comprising the steps of:
generating of laser pulses with a pulse duration of up to 1 ps at a repetition frequency of at least 1 MHz, preferably at least 10 MHz,
converting the laser pulses into THz pulses by means of a first THz antenna serving as sender, and
receiving the THz pulses by means of a second THz antenna serving as receiver. In accordance with the invention, the second THz antenna is coupled to a detector circuitry whose bandwidth is at least equivalent to the repetition frequency of the laser pulses, with recording the chronological progression of the amplitudes of the THz pulses received consecutively by the second THz antenna.

Therefore, in other words, the key idea of the present invention is utilizing a detector circuitry for incoherent detection of THz radiation, the time resolution of which is so rated that the amplitude of each individual THz pulse received by the second THz antenna can be detected. According to the present invention, each individual THz pulse is resolved by the compound comprised of THz antenna and detector circuitry. Measurement of the temporal change of the amplitude from pulse to pulse, for example, thus allows for investigating dynamics which generate a quickly variable absorption of the THz radiation in an irradiated sample.

By means of an appropriate evaluation unit linked to the detector circuitry, the present invention allows for recording, i.e. pulse by pulse, the cronological progression of the amplitudes of THz pulses consecutively received by the second THz antenna. Unlike prior art coherently measuring terahertz systems, the inventive incoherent measurement does not require optical or electronic coupling of the THz antenna and detector circuitry to the laser light source. Accordingly, the inventive terahertz system has no adjustable delay of light pulses as are required for conventional systems.

Since the detector circuitry according to the present invention has a bandwidth which is at least equivalent to the repetition frequency of the laser light source, the time resolution achievable according to the present invention corresponds to the pulse distance of the laser pulses, i.e. the inverse repetition frequency. A time resolution in a range of below 10 ns is thus achievable. As compared with the state of the art, this corresponds to an improvement by 5-6 orders of magnitude. Correspondingly faster can a measurement be taken according to the present invention. This opens-up absolutely new fields for application of time-resolved terahertz measuring technology.

With a preferred embodiment of the inventive terahertz system, the laser light source is an erbium-doped mode-locked fiber laser. Such a fiber laser is commercially available at low cost. For example, one can use a laser light source that is usually implemented in telecommunication applications. The fiber laser can be coupled via a suitable optical fiber to the first THz antenna. Usual erbium-doped mode-locked fiber lasers generate light pulses with a pulse duration of some 100 fs at a repetition frequency in a range of 50-100 MHz. They are well suited as laser light sources for the inventive terahertz system.

With a further preferred embodiment of the inventive terahertz system, the first THz antenna is a photoconductive antenna comprised of an antenna structure subjected to electrical pretension and being electrically conductive on a semiconductor substrate. THz antennae of this type are commonly applied to generating THz radiation. Especially suitable for the present invention, however, is an embodiment in which the semiconductor substrate comprises a multiple-layer structure, whereof at least one layer is comprised of doped or undoped InGaAs and whereof at least another layer is formed by doped or undoped InAlAs, InGaAsP or InGaAlAs. Such a photoconductive antenna is especially suitable for application in accordance with the present invention, because the light pulses of the laser light source are converted with high efficiency into THz pulses. Thereof, it results a high THz output. Consequently, one can dispense with a specially sensitive and thus costly detection technology (e.g. lock-in amplifier). Suitable THz antenna, for example, are disclosed in DE 10 2010 049 658 A1 in which the electrically conductive antenna structure is integrated on the semiconductor substrate which consitutes an InAlAs/InGaAs-multiple-layer heterostructure (see Applied Physics Letters 103, 061103, 2013).

In accordance with the present invention, it is important, as has been outlined above, that the THz radiation is detected in broadband manner so that the measurement of the THz pulses has sufficient time resolution. In terms of the signal-to-noise ratio, this requires the THz pulses to have adequate mean power of preferably at least 1 μW, further preferred at least 10 μW, particularly preferred at least 50 μW. This can be achieved by utilizing a photoconductive antenna of the type described hereinabove as a first THz antenna in the sense of the present invention. A broadband detection in the sense of the present invention particularly excludes applying a lock-in technology or a similar narrow-band detection method. The inventive terahertz system therefore distinguishes itself in that the detector circuitry preferably has no phase-sensitive equalizer nor a substrate frequency amplifier nor a narrow-band bandpass filter which usually are components of lock-in detectors.

With a further preferred embodiment of the inventive terahertz system, the detector circuitry comprises a Schottky diode which is connected with the THz antenna. Schottky diodes can be realized as semiconductor circuitry elements working with particularly high frequency. Working frequencies up and into the range of terahertz are possible, in particular without a pretension pending at the Schottky contact (“zero bias”). Thereby it is possible within the sense of the present invention to detect the amplitudes of individual THz pulses, i.e. with adequate speed, so that the individual THz pulses can be resolved. Particularly suitable is a Schottky diode in which a metallic layer contacts a semiconductor layer by formation of the Schottky contact, a plastic layer embedding the Schottky contact and forming a substrate for the semiconductor circuitry element. With such a Schottky diode, a detector circuitry working in the terahertz range can be realized according to the present invention. Receivers based on corresponding Schottky diodes have recently become commercially available (for example from the producer ACST Advanced Compound Semiconductor Technologies GmbH, Darmstadt). EP 2 528 090 A1 discloses further details on the set-up of an appropriate Schottky diode and in relation to realizing a THz detector circuitry on that basis.

In a feasible embodiment, the detector circuitry may comprise a resonant circuit with capacitor and inductivity which is harmonized to the frequency of the THz radiation. Then, the Schottky diode is integrated into the resonant circuit in order to convert the amplitudes of THz pulses received into electrical signals.

The inventive terahertz system is suitable for non-destructive testing of an object, for instance for process monitoring or quality control. Accordingly, THz pulses emitted from the first THz antenna irradiate the object to be examined, wherein the THz pulses reflected from the object and/or transmitted through the object are received by the second THz antenna. On account of the high measuring speed, the object under examination can quickly move relatively to the sender and receiver of the terahertz system. A movement speed of several meters per second is possible. Likewise, because of the high measurement speed, larger measuring surface areas can be covered, e.g. by scanning several measuring points on the area consecutively by means of the THz radiation.

Furthermore, the inventive terahertz system is suitable for THz imaging, moving the arrangement comprised of the first and second THz antenna relative to an object to be imaged, in particular around the object. THz imaging, too, benefits from the high measuring speed of the inventive terahertz system. The arrangement comprised of the first and second THz antenna (sender/receiver) can be moved at high speed along and/or around the object to be imaged in order to record the required image dataset completely within the shortest time.

Further fields of application of the inventive terahertz system are to be mentioned in the following:

The absorption of THz radiation depends on the moisture content of the object to be examined. Hence, the inventive system is suitable for measuring the moisture content in plastics, food or paper. The grammage of paper, e.g. for quality control in paper manufacture, can be measured. Thereby, beta emitters hitherto used for this purpose can be replaced. Likewise, the moisture content in plant leaves can be measured, e.g. for optimizing irrigation strategies. The chronological progression of drying processes can be monitored in accordance with the present invention. The absorbency, e.g. of cellulose (diapers) can be investigated. THz imaging can be implemented for detection of tramp material, inclusions, air bubbles, delamination and other defects. Layer thickness measurements of THz transparent materials can be carried out, for example in the production of tubes, plates or foils made of plastic. In biological systems, fast chemical processes can be investigated and monitored, e.g. folding dynamics of biomolecules. Such processes which cannot be investigated by applying conventional pump-probe methods, e.g. since those processes of interest cannot be excited optically and/or repeated periodically, are well accessible to investigation by applying the inventive system. The inventive system and the method are furthermore suitable for use as tools for developing, testing, and characterizing fast THz optics, e.g. on the basis of liquid crystals. Finally, THz measurements are made possible by the present invention under quickly varying ambient conditions, e.g. varying magnetic fields or temperatures.

A practical example of the present invention is explained in greater detail in the following by way of drawings, where:

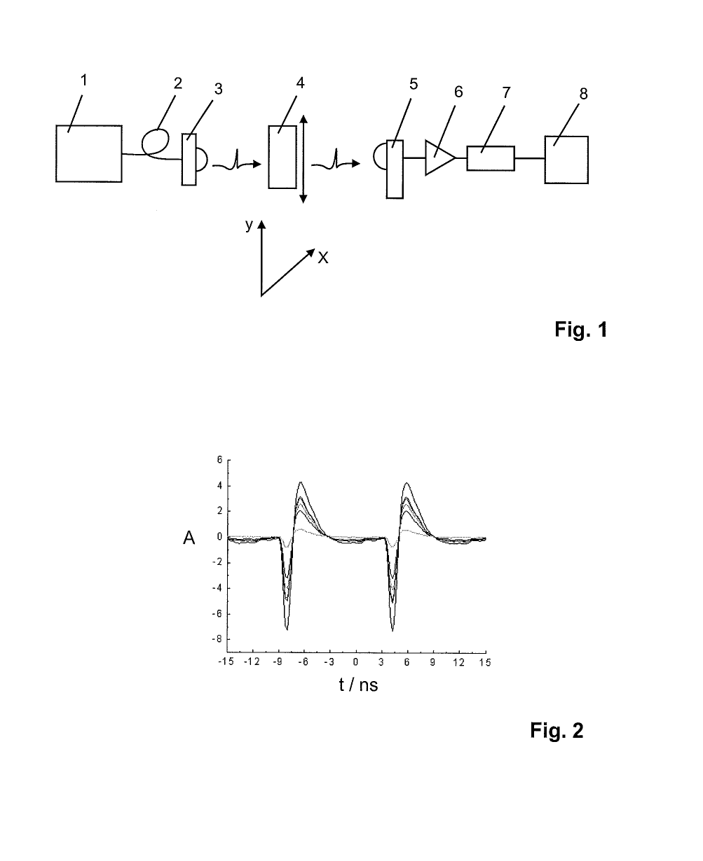

FIG. 1: is a block-type diagram of an inventive terahertz system;

FIG. 2: illustrates a time-resolved measurement of THz pulses according to the present invention.

FIG. 1 shows an inventive terahertz system in form of a block-type diagram. It serves for generating and incoherent detection of THz radiation. The system comprises an erbium-doped fiber laser 1, which generates laser pulses with a pulse duration of approx. 100 fs at a repetition frequency of 80 MHz. The laser pulses are passed via a light-conducting fiber 2 to a sender 3. The latter comprises a first THz antenna in form of a photoconductive antenna (not illustrated), featuring an antenna structure subjected to electrical pretension and being electrically conductive, and integrated on a semiconductor substrate. The semiconductor substrate comprises a multiple-layer InAlAs/InGaAs structure. The photoconductive antenna converts the light pulses of the femtosecond laser 1 with high efficiency into THz pulses, i.e. each laser pulse generates one THz pulse. With this practical example, intensive THz pulses in a 12 ns time interval are thus generated. The THz pulses generated have a pulse duration of typically some ps. Their spectrum contains frequencies of approx. 50 GHz-4 THz, the maximum ranging between approx. 300-500 GHz. The THz pulses radiate through a sample 4, which in this practical example is moved in y-direction. The THz pulses transmitted are received by a receiver 5 which comprises a second THz antenna (not illustrated). The second THz antenna which is not coupled optically to the femtosecond laser 1 is connected to a detector circuitry which comprises a Schottky diode (not illustrated). High measuring speed is achieved by the combination of the photoconductive antenna in the THz sender 3 and the (“zero bias”) Schottky diode in the THz receiver 5. The output signal of receiver 5 is amplified by means of an amplifier 6, and converted by means of a digital/analogous converter 7 into a digital signal. By means of an evaluation unit 8, e.g. in form of a personal computer, the chronological progression of the amplitude of the THz pulses consecutively received by receiver 5 is recorded.

In the practical example shown here, the THz receiver 5, comprised of the zero bias Schottky diode is sensitive between 0.05 THz and 1.5 THz. Though the bandwidth of receiver 5 thus does not reach the entire bandwidth of sender 3, but it covers a sufficiently large area and above all that area in which the spectrum of sender 3 reaches its maximum. Receiver 5 is connected to a transimpedance amplifier 6 having a bandwidth of 4 GHz. In combination with adequately fast data acquisition electronics, the system is capable of measuring and displaying each individual THz pulse. FIG. 2 shows the digitalized and recorded output signals of receiver 5. Curves shown here represent an air reference signal and/or signals at which the THz pulses have passed through differently thick plastic materials. The diagram shows the output signal A (arbitrary units) of receiver 5 as a function of time in ns. The diagram demonstrates that the individual THz pulses are well resolved in terms of time and can be evaluated in terms of their amplitude. The time resolution lies in a range of some ns.

1. A terahertz system for generating and time-resolved incoherent detecting of THz radiation, said system comprising
a pulsed laser light source (1) which emits laser pulses with a pulse duration of up to 1 ps at a repetition frequency of at least 1 MHz, preferably at least 10 MHz,
a first THz antenna serving as sender (3), said antenna coupled optically to the laser light source (1) and converting the laser pulses into THz pulses; and
a second THz antenna serving as receiver (5),
wherein the second THz antenna is coupled to a detector circuitry whose bandwidth is at least equivalent to the repetition frequency of the laser light source.

2. The terahertz system according to claim 1, wherein the second THz antenna and the detector circuitry are not coupled optically or electronically to the laser light source (1).

3. The terahertz system according to claim 1, wherein the detector circuitry comprises neither a phase-sensitive equalizer, nor a substrate frequency amplifier, nor a narrow-band bandpass filter whose filter bandwidth is smaller than the repetition frequency of the laser light source.

4. The terahertz system according to claim 1, characterized by an evaluation unit (8) linked to the detector circuitry, said evaluation unit being equipped to record the chronological progression of the amplitudes of the THz pulses consecutively received by the second THz antenna.

5. The terahertz system according to claim 1, wherein the laser light source (1) is an erbium-doped mode-locked fiber laser.

6. The terahertz system according to claim 1, wherein the first THz antenna is a photoconductive antenna comprising an antenna structure on a semiconductor substrate, said antenna structure being subjected to electrical pretension and being electrically conductive.

7. The terahertz system according to claim 6, wherein the semiconductor substrate comprises a multiple-layer structure, whereof at least one layer is formed from doped and undoped InGaAs, and whereof at least another layer is formed from doped or undoped InAlAs, InGaAsP or InGaAlAs.

8. The terahertz system according to claim 1, wherein the detector circuitry comprises a Schottky diode connected to the THz antenna.

9. The terahertz system according to claim 8, wherein the Schottky diode is a semiconductor circuitry element in which a metallic layer contacts a semiconductor layer by formation of a Schottky contact, wherein a plastic layer embeds the Schottky contact and forms a substrate for the semiconductor circuitry element.

10. The terahertz system according to claim 8, wherein the detector circuitry comprises a resonant circuit with capacitor and inductivity.

11. A method for generating and time-resolved incoherent detecting of THz radiation, said method comprising the steps of:
generating of laser pulses with a pulse duration of up to 1 ps at a repetition frequency of at least 1 MHz, preferably at least 10 MHz,
converting the laser pulses into THz pulses by means of a first THz antenna serving as sender (3), and
receiving the THz pulses by means of a second THz antenna serving as receiver (5),
wherein the second THz antenna is coupled to a detector circuitry whose bandwidth is at least equivalent to the repetition frequency of the laser pulses, wherein the chronological progression of the amplitudes of the THz pulses consecutively received by the second THz antenna is recorded.

12. Use of a terahertz system according to claim 1 for non-destructive testing of an object (4), wherein the THz pulses emitted from the first THz antenna irradiate the object (4) to be examined, wherein the THz pulses reflected from the object (4) and/or transmitted through the object (4) are received by means of the second THz antenna.

13. Use according to claim 11, wherein during testing the object is moved relative to the sender (3) and receiver (5) of the terahertz system.

14. Use of a terahertz system according to claim 1 for THz imaging, wherein the arrangement of a first and a second THz antenna is moved relative to an object to be imaged, more particularly rotated around the object.

15. Use of a terahertz system according to claim 1 for investigating the kinetics of chemical processes, more particularly of folding dynamics of biomolecules.
